(19) United States
(12) Patent Application Publication
(10) Pub. No.: US 2001/0051351 A1
(43) Pub. Date: Dec. 13, 2001

(54) Antigen-specific immune complex-based enzyme-linked immunosorbent assay

(76) Inventor: Stanley Paul Racis, North Sioux City, SD (US)

Correspondence Address: KIRKPATRICK & LOCKHART LLP, 535 SMITHFIELD STREET, PITTSBURGH, PA, 15222, US

(21) Appl. No.: 09/816,271

(22) Filed: Mar. 23, 2001

Related U.S. Application Data
(60) Provisional application No. 60/192,472, filed on Mar. 27, 2000.

(51) Int. Cl. G01N 33/574; G01N 33/569

(52) U.S. Cl. 435/7.33; 435/7.23

(57) ABSTRACT

The invention is in the field of immunologic serological in vitro diagnostics. The invention is an ELISA-based diagnostic testing system and method that provides the capability to “look within” and measure an immune complexes specific antigen and antibody using typical ELISA microplates and procedures. One aspect of the invention is a method for detecting antigen and antibody in immune complexes. A second aspect of the invention is for a well design that may be used in the method of the invention. A third aspect of the invention is for a kit for detecting antigen, antibody, or both antigen and antibody in immune complexes.

### CROSS-REFERENCE TO RELATED APPLICATIONS

This application claims priority under 35 U.S.C. §119(e) from U.S. application Ser. No. 60/192,472, filed Mar. 27, 2000.

### BACKGROUND OF THE INVENTION

An individual's immune system responds to foreign matter or antigens (Ag) by synthesizing specific antibodies (Ab) that can interact with initiating antigens and non-covalently bind the antigens to form antigen-antibody complexes or immune complexes (IC). The normal immune response of antibody binding to antigen to form an immune complex usually benefits the host by eliminating and/or neutralizing the antigen. When an immune complex is found as a soluble component of bodily fluids, it is known as a circulating immune complex (CIC).

It has been known for years that circulating immune complexes are relevant to a large number of diseases. More recently, in vivo and in vitro experiments have helped elucidate many of the factors involved in immune complex formation, removal and localization, as well as the mechanisms of immune complex induced inflammatory reactions. The current belief is that immune complexes can be potentially pathogenic, as well as regulate both cellular and humoral immune responses by interacting with antigen receptor bearing lymphocytes, subpopulations of T and B cells, and other immune and non-immune cells. With the recognition of the immunopathologic consequences of immune complexes and the development of new techniques for demonstrating immune complexes in tissues and biological fluids, considerable evidence has been accumulated substantiating the primary pathogenic significance of immune complexes in a variety of animal and human diseases. It is now understood that circulating immune complexes in man and lower animals are responsible for, or associated with, a diverse array of diseases. These diseases include autoimmune diseases, such as rheumatoid arthritis and systemic lupus erythematosus (SLE); neoplastic diseases, such as leukemia, ovarian cancer and breast cancer; infectious diseases due to bacteria, viruses and parasites; and other unclassified disorders. Furthermore, both exogenous and endogenous antigens can trigger pathogenic immune responses, resulting in immune complex disease.

The awareness of the important role of immune complexes in many diseases has stimulated the development of techniques for demonstrating their presence in tissues and biological fluids. Historically, the development of assays for soluble immune complexes involved physiochemical techniques. However, immunological techniques are more commonly used today.

The basis for many immunoassays is similar, the binding of an antigen or hapten to a specific antibody. To facilitate measurement, a ligand, usually antigen or antibody, is displayed in solution or on some form of solid support, such as cell membranes, microscope slides, micro-titer wells, or diffusion or electrophoretically separated bands that are stabilized in gels. The fluid to be tested, usually blood, urine, cerebrospinal fluid or cell cultures, is then placed in contact with the solution or solid support that is displaying the ligand. If analyte, the substance to be measured, usually antibody or antigen, is present in the test fluid, it will bind to the solid support via the ligand display. Unbound materials are washed free of the solid support. Most often, but not necessarily, a secondary antibody with an attached signal-emitting molecule, such as fluorochromes, enzymes, radioisotopes, magnetic substances, or chemiluminescent compounds, is incubated with the solid support to facilitate measurement of analyte binding to the ligand. After incubation, unbound labeled secondary antibody is washed free of the solid support. Finally, quantitative detection of the signal is performed to determine the concentration of analyte in the fluid sample.

One type of immunoassay is a sandwich assay. Although there are many variations of this technique, the basic concept is simple. First, an antibody that is specific to an analyte of interest is immobilized on a solid-phase support. Next, a sample that potentially contains the analyte is incubated with the bound antibody. Then, a second antibody, which reacts with the analyte at a different site than that of the bound antibody, is added to the mixture. Detection of the second antibody is then used to measure the amount of analyte in the sample.

Another commonly used immunological technique is an enzyme-linked immunosorbent assay (ELISA). ELISA is an enzymatic variation on the sandwich assay described above. However, the goal of an ELISA is to detect an antibody of interest so the roles of binder and ligand are reversed. Therefore, in an ELISA, antigen is bound to a solid-phase substrate. A sample containing an antibody of interest is incubated with the bound antigen. Then, a second, enzyme-labeled antibody directed against the antibody of interest is added to the mixture. Next, an enzyme substrate and any necessary buffers are added so that the enzyme may metabolize the substrate to form a reaction product. Finally, measurement of the reaction product is used to detect the quantity of antibody of interest in the sample.

Currently used methods for identifying antigen specific components of circulating immune complexes are sodium dodecyl sulphate polyacrylamide gel electrophoresis (SDS-PAGE), staged chromatographic methods, multiple discrete assays, or other such equipment that is both sample and labor intensive. Therefore, there is a need for a detection method that is more rapid, reliable and cost effective than the presently available detection methods and test kits.

### BRIEF SUMMARY OF THE INVENTION

The invention is in the field of immunologic serological in vitro diagnostics. The invention is an ELISA-based diagnostic testing system and method that provides the capability to “look within” and measure a captured circulating immune complex's specific antigen and antibody using typical ELISA microplates and procedures. The system and method of the present invention are more clinically relevant than measuring either antigen or antibody alone, and are useful for the host of diseases and conditions for which markers for the disease or condition have been identified. The technology can also be used to elucidate and/or screen for the humoral immune response's targets within selected individuals, groups of individuals with shared diseases or conditions, and microarray data suggesting sets of activated genes or altered proteomic profiles.

One aspect of the invention is a method, and some variations on the same, for detecting antigen and antibody in immune complexes. The method of the present invention comprises: capturing a circulating immune complex, resulting in a captured immune complex; dissociating the captured immune complex; re-associating the captured immune complex with an appropriate reference material, such as labeled antigen, antibody, and/or immune complexes (IC), to form a reformed immune complex; and detecting and quantitating the reference material, and hence antigen, antibody, or both antigen and antibody in the reformed immune complex.

A second aspect of the invention is a well design that may be used in the method of the invention. The well design has several important characteristics. The first characteristic of the well is that it has one or more surface area increasing members, such as vertical fins rising from the well bottom, to increase the wells' overall functional surface area. Another characteristic is that the well is designed to remain substantially optically transparent in the appropriate substrate buffer.

A third aspect of the invention is a kit for detecting antigen, antibody, or both antigen and antibody in immune complexes.

### BRIEF DESCRIPTION OF THE DRAWINGS

The following drawings are for illustrative purposes to facilitate better understanding of the invention and not for limiting the same.

FIG. 1 is a flowchart of the core steps of the method for detecting antigen, antibody, or both antigen and antibody in immune complexes.

FIG. 2 is a flowchart of an embodiment of the method for detecting antigen, antibody, or both antigen and antibody in immune complexes.

FIG. 3 is a top plan view of the well of the present invention.

FIG. 4 is a side elevation view of the well of the present invention.

### DETAILED DESCRIPTION OF THE INVENTION

The present invention provides a method for detecting antigen and antibody in immune complexes, a well design particularly adapted for use in the method of the invention, and a kit for detecting antigen and antibody in immune complexes. The method is an ELISA-based diagnostic testing method. The method provides the capability to “look within” and measure an immune complex's specific antigen and antibody within both intact and dissociated immune complexes using typical ELISA microplates and procedures.

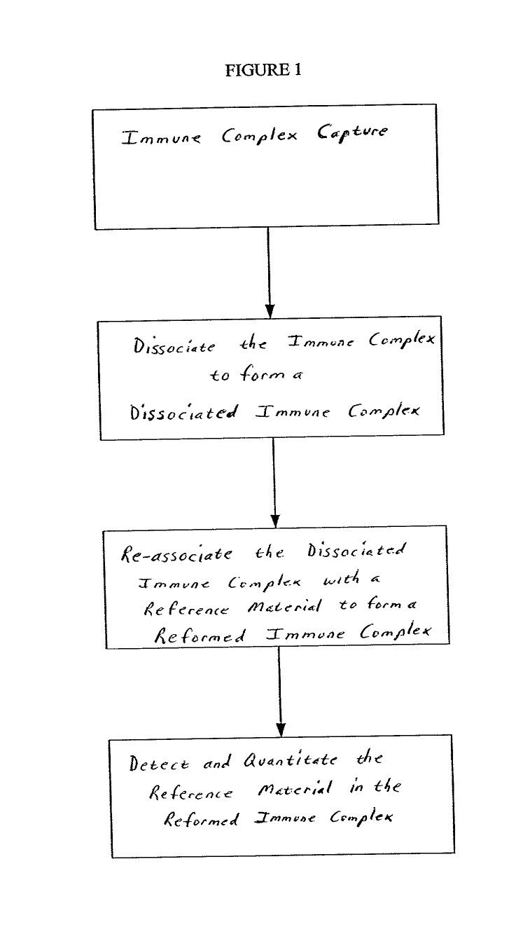

The method of the present invention comprises the core steps of: capturing a circulating immune complex, resulting in a captured immune complex; dissociating the captured immune complex; re-associating the captured immune complex with an appropriate reference material, such as labeled antigen, antibody, and/or immune complexes (IC) to form a reformed immune complex; and detecting and quantitating the reference material, and hence antigen, antibody, or both antigen and antibody in the reformed immune complex, as shown in FIG. 1. Immune complexes may be captured by any appropriate means for the disease or disorder in question. Immune complexes are preferably dissociated using agents that cause high salt concentration or low pH, however other methods are also possible. Immune complexes may be re-associated using agents that decrease the salt concentration or increase the pH of the mixture, however other methods are also possible. Finally, detection and quantitation may be accomplished by measuring the optical density of the sample, although other methods of detection and quantitation are possible.

The method of the present invention can be used to detect antigen, antibody, or both antigen and antibody for use in diagnosing numerous diseases. For example, the method of the invention can be used to detect proteins that the immune system recognizes as important in disease. Such proteins include, but are not limited to, coagulation proteins and tumor derived proteins, such as growth factors and their receptors, gene regulation factors, proteins that are important for the development of metastasis, and angiogenic proteins.

The first core step of the method is to capture one or more circulating immune complexes from a sample in an initial capture, resulting in one or more captured immune complexes. Capture simply means to selectively bind the sought for type or subtype of circulating immune complex to a solid phase component. Examples of solid phase components are wells, gels, polyurethane, polystyrene, magnetic beads or a metallic substrate. Binding to the solid phase can occur by any one of or a combination of the following molecular forces: electrostatic interactions, ionic bonding or covalent bonding. Preferred sample sources are blood, tears, saliva, lymph and urine. However, other sample sources may be used. Circulating immune complexes are captured by any appropriate means for the disease or disorder in question, such as in the reference by Lawley, “Methods of Detection of Circulating Immune complexes”, Clinical Immunology & Alert, vol. 1, pp. 383-396 (1981), which is incorporated herein by reference. The actual capture system will be dependent upon the particular application in terms of whether immunoglobulin (Ig), antigen, complement factors or other immune complex associated molecules are of interest. In general, one or more of the generally accepted means of immune complex capture may be used, including a capture agent. A capture agent is an agent such as a molecule, chemical, receptor, protein or antibody, that is used to bind the sought for type or subtype of circulating immune complex to a solid phase component. Some of the acceptable capture agents include, without limitation, Staphylococcal proteins, complement component Clq, and anti-complement antibodies.

For example, if complement activation is the pathological marker desired, then capture of complement containing immune complexes, Clq or C3 products, would be appropriate. In addition, if the interaction of antigen and antibody is the target, then Staphylococcal proteins such as protein A or protein G, or antibody directed against human antibody could be used. Thus, the exact composition and nature of the capture phase is dependent upon the particular application with regard to the type or subtype of circulating immune complex deemed most clinically relevant for the disease or disorder.

The capture system is important to the present invention. However, some variables of the capture system are application specific. For example, if immunopathology involving complement-containing circulating immune complexes is important, then circulating immune complexes displaying complement would be important in terms of the capture system. However, if disease or tumor markers within circulating immune complexes are important, then the capture system would be designed to capture all possible circulating immune complexes rather than just subsets of circulating immune complexes.

As stated above, circulating immune complexes are captured by any appropriate means for the disease or disorder in question. In most cases, the appropriate means for capturing circulating immune complexes can be readily determined in each case by one of ordinary skill in the art by reference to the literature, using particularly known markers for specific diseases or disorders. For example, in systemic lupus erythematosus, DNA is the predominant marker of choice, and complexes containing DNA and anti-DNA that are capable of fixing and activating complement, because of their inherent ability to lead to immunopathology, would probably dictate the use of a complement based capture system comprising Clq or the C3 products.

If the appropriate means for capturing circulating immune complexes is not available in the literature, the present invention also provides a method for determining the appropriate capture system for a particular patient's circulating immune complexes. The method comprises pre-screening samples from patients along with appropriate controls for different “types” of circulating immune complexes. The pre-screening tries to detect the presence of elevated circulating immune complex levels in different ELISA capture systems, such as systems based on Clq, Staphylococcal proteins, C3, Raji cell membranes, conglutinnin and MC antibodies. For example, the types of circulating immune complexes can be divided into groups: those which can and/or have bound Clq, those which can or have activated C3, and those which may or may not have activated or bound any complement. Next, the circulating immune complexes are captured with various capture systems for blot analysis, as described in Johnstone & Thorpe, “Immunochemistry in Practice”, which is incorporated herein by reference. Using blot analysis one can identify the individual blots or bands in terms of known markers, potential gene products from microarray data and/or other genetic analysis, suspected markers, or biochemical identification. The result is identification of the circulating immune complex components in each case. Statistical analysis is then performed for correlation of any of the circulating immune complexes type's components with the appropriate clinical parameters for that disease. Finally, the user can choose the capture method or methods that are significantly able to capture circulating immune complexes that correlate with the disease's clinical parameters. This procedure enables the user to determine the appropriate capture conditions for the capture phase.

The capture of circulating immune complexes may involve the use of a solid substrate or carrier, such as micro-titer wells. A carrier is any molecule or molecular conglomerate that can be utilized as an intermediary to bind circulating immune complexes and facilitate removal from the liquid phase, such as by aggregation or precipitation. The invention comprises a novel well design that will be discussed in greater detail below. For now, it should be realized that the choice of an appropriate well should allow for deposition/attachment of the capture agent or material without disrupting the optics of the well. The preferred method for depositing the capture material on the well is lyophilization. Wells made of immulons, polystyrenes or polyethylenes all work well in the method of the present invention. However, in order to better control and maximize recovery, the user may pre-treat the wells with a commercially available cross-linker. A cross-linker is any chemical or substance used to facilitate the attachment to the solid phase of the molecule that captures the circulating immune complex. The use of a cross-linker is preferred. Some examples of commercially available cross-linkers are poly-L-lysine, gluteraldehyde and cyanogen bromide. An acceptable method of pre-treating the wells is described in Voller et al., “The Enzyme Linked Immunosorbent Assay (ELISA)”, Dynatech Labs, catalogue no. 011-010-6200.

In the method of the present invention, immune complexes exist in either a state of antigen excess, antibody excess or equivalence. Therefore, all three possibilities must be taken into consideration. The method of the invention follows standard ELISA procedures where multiple wells are employed when assuming the captured immune complexes are in antigen excess, antibody excess or equivalence. Simple interaction of the captured immune complex with appropriately labeled antigen or antibody will result in a qualitative determination for the specific presence of target antigens.

To test equivalent circulating immune complexes and/or to determine the amount of specific moiety from the first two conditions, the circulating immune complexes are dissociated and then re-associated with enzyme labeled markers that are added in a carefully controlled fashion.

The next core step of the invention involves dissociating the captured immune complex, resulting in a dissociated immune complex. The dissociation stage is application dependent so the particular dissociation conditions will depend upon the particular subtype of circulating immune complex that was captured. However, agents that cause high salt concentrations or low pH have proven to be effective dissociation agents, with alteration of the pH most preferable. In order to dissociate the captured immune complexes using high salt concentration, the minimum salt concentration should be approximately 300 mM NaCl while the maximum salt concentration should be approximately 1.5 M NaCl. However, 500 mM NaCl is most preferred. The preferred salt solution has a pH of approximately 7.2 and comprises either 500 mM NaOH, 2 mM EDTA and 50 mM Tris buffer, or 500 mM NaOH, 2 mM EDTA and 50 mM sodium phosphate. The problem with altering the salt concentration is that it makes the re-association stage more difficult in that it requires more time and more wells. Alternatively, the captured immune complexes can be dissociated with low pH. The effective pH maximum for the dissociation stage is approximately 2.8. The preferred pH range is 1.5 to 2.5. The most preferred pH range is 2.0 to 2.5. However, it should be realized that the lower the pH, the shorter the dissociation time that is needed. Acids, such as acetic acid, citric acid and hydrochloric acid, are preferable for lowering the pH.

In addition to either raising the salt concentration or lowering the pH, other methods of dissociating immune complexes are also possible. These other methods may be used if either raising the salt concentration or lowering the pH causes the sought after component to become denatured or causes some other physical change or problem that makes the re-association or detection and quantitation steps more difficult or impossible. These other methods include: sonication at over 130 Kc/sec for 30 minutes or more, as disclosed in Grabar et al., Adv. in Biol. & Med. Phys., vol. 3, pp. 191-208 (1953); changes in osmolarity; and SDS, as disclosed in Johnstone & Thorpe, “Immunochemistry in Practice”, Blackwell Scientific Publications, pp. 155-173 (1982); all of which are incorporated herein by reference. In addition, a general reference for dissociation techniques is Johnstone & Thorpe, “Immunochemistry in Practice”, Blackwell Scientific Publications, chapters 6, 7 & 10 (1982), which is incorporated herein by reference.

In addition to either raising the salt concentration or lowering the pH, the dissociation conditions should occur for a short period of time and include bovine serum albumin (BSA). Several types of BSA may be used, with 1 % RIA-grade BSA generally being sufficient. The short period of time for dissociation by lowering the pH is application dependent, but 5 to 30 seconds is generally optimal. However, each application's specific antigen or marker of interest might require some modification to the time if the antigen or marker proves too sensitive to the lower pH. The maximum time for dissociation has been observed to be 4 to 5 minutes for most glycoproteins, including antibody. However, significant loss of efficiency has been observed to occur generally after 1 minute of exposure to dissociating conditions.

After the dissociation step, an optional intermediate step may be performed. The intermediate step involves adding a metered amount of labeled antigen or antibody so that one can perform a competitive ELISA analysis for the amount of unlabeled antigen or antibody within the originally captured materials. For example, enzyme-labeled antibody is used to target internal antigens and/or markers within the circulating immune complex. On the other hand, enzyme labeled antigen is used when antigen specific antibody is itself the specific target being detected. It is generally preferred that the labeled materials be added in conjunction with the re-association phase.

The competitive ELISA of the optional intermediate step takes place in the previously prepared capture wells. It is preferred that the dissociating solution is in a volume at least 4 fold smaller than the re-associating solution. However, the dissociating volume is preferably not less than 10 microliters and not greater than 50 microliters. Re-associating volumes are limited only by the volume of the well. It is most preferred to have a dissociating volume of 20 microliters and a re-associating volume of 20 to 130 microliters. It is desireable to minimize the re-association volume, as a larger re-association volume usually requires increased times for re-association.

Following or with the optional intermediate step, if it is used, the invention includes the core step of re-associating the dissociated immune complex with a reference material. Preferably, the re-association step is coincident with the addition of labeled reference materials. A reference material is any material that is labeled with a marker and that competitively binds the desired component of the dissociated immune complex. Some examples of reference materials are enzyme-labeled markers, such as labeled antigen, antibody, or immune complexes, that are added in a carefully controlled fashion to form a reformed immune complex. Such re-association methods include re-associating agents that decrease the salt concentration or increase the pH in the mixture. However, re-association may involve simply reversing the dissociation conditions in the original well. Alternatively, the salt concentration could be decreased by dilution, or excess salt could be removed by dialysis. On the other hand, if the dissociation step involved lowering the pH, then the re-association step will involve increasing the pH. Therefore, the buffer should overcome the low pH from the previous dissociation step and readjust the pH to approximately 7.2 with variable protein loads.

For example, one can initially use unbuffered acid to drop the pH (for dissociation), putting it in as small a volume as possible, then raise the pH back again with buffers such as PBS or carbonate buffer (for re-association). Alternatively, re-association may require transfer of the dissociated materials to another prepared well as a function of the specific antigens involved. For example, if the antigen is not a protein, such as sterols or nucleic acids, different buffers and solvents might be required which would necessitate a non-aqueous phase for re-association and/or secondary capture.

A parameter in the method of the present invention is the time of incubation for re-association. The incubation time for re-association is dependent upon the specific antigens in question and can vary from as low as 20 minutes to as high as 12 or more hours, at differing temperatures. Typically, incubation is for 20 minutes to 2 hours, and preferably 1-2 hours, at room temperature. Re-association should occur for at least 20 minutes, and 60 minutes is preferable. However, if known low affinity reactions are involved, it may be preferable to incubate at 4-10° C. for 8-12 hours.

Another variable for the re-association stage is the amount of enzyme-labeled marker needed for maximum sensitivity of sample antigen/antibody marker interference. This is determined as follows. Typically, enough enzyme-labeled antibody or antigen is delivered with the re-association buffer so as to be in 10-200 fold excess of the maximum optical density (OD) reading possible for the enzyme-substrate system employed. Any enzyme labeled material not complexing with its ligand is subsequently washed away during the capture phase. Standard reference preparations are determined by generating a standard dose response curve for titrated unlabeled target materials for each of a wide range of enzyme-labeled reactants. Typically, the highest concentration of enzyme-labeled reactants which do not result in background readings (when no unlabeled ligand is present in a sample) above 0.100 are employed. By titrating known amounts of unlabeled target antigen or marker with this concentration of enzyme-labeled reactant, a typical control curve is generated which allows for quantitative determinations.

The next core step of the invention involves detecting and quantitating the reference material, and hence, the antigen, antibody, or antigen and antibody within the reformed immune complexes. Detection and quantitation occurs after the labeled and reformed circulating immune complexes are bound to the solid phase. The samples are then washed, the appropriately buffered colorless substrate is added and an optical density (OD) reading of the substrate's color intensity is taken using standard commercially available readers. The OD readings are interpreted using a standard curve as a reference. Alternatively, in the competitive ELISA embodiment of the present invention, the degree of inhibition of control enzyme labeled binding relative to controls quantitatively reflects the amount of specific antigen in the original sample.

Another embodiment of the invention involves a secondary capture (or recapture) or subsequent specific antigen capture. The secondary capture refers to a capture other than the initial capture of circulating immune complex and occurs after the initial capture step. The preferred method of secondary capture is to utilize the same capture system in the same well as the initial capture, but with an antigen-specific labeled antibody that overwhelms the unlabeled aspect, i.e. the antigen of interest in the circulating immune complex, during re-association. However, individual applications may warrant the direct capture of the specific antigen, either in the same well or in other wells, and/or employing secondary capture systems on other surfaces, such as a well surface, dipsticks, beads or membranes. As stated above, the secondary capture may proceed in either the same well as the initial capture or in a newly prepared well. If the secondary capture proceeds in the same well as the initial capture, then no secondary capture preparations are needed. However, if the secondary capture proceeds in a newly prepared well, then secondary preparations are needed, based upon the specific antigen or antibody sought. Some acceptable wells for the secondary capture phase include: EB plate, manufactured by Labsystems Co., Ltd. (Atlanta, Ga.); H type plate, manufactured by Sumitomo Bakelite Co., Ltd (Singapore); C type plate, manufactured by Sumitomo Bakelite Co., Ltd (Singapore); and Maxi-Soap plate, manufactured by Nunc Co., Ltd (Naperville, Ill.).

For example, if the secondary capture is targeting anti-Beta 2-Glycoprotein I, the following procedure could be employed in a series of wells. Beta 2-Glycoprotein I is coated to the desired carrier. Beta 2-Glycoprotein I may be bound to the carrier by appropriately combining and conducting conventional processes under suitable conditions known for immobilization of a protein, such as an enzyme, as discussed in: Liang et al., “Biomedical application of immobilized enzymes”, J Pharm. Sci., vol. 89(8), pp. 979-990 (2000); Wan et al., “Behavior of soluble and immobilized acid phosphatase in hydo-organic media”, Biochim Biophysca Acta, vol. 20:410(1), pp. 135-144 (1975); and Baess et al., “Comparison of the activities of free and carrier fixed horseradish peroxidase”, Acta Biol. Med. Ger., vol. 34(6), pp. 965-969 (1975); which are all incorporated herein by reference. For example, any technique including physical adsorption, ionic binding, or covalent bonding may be used.

Some examples of carriers are EB plate, H type plate, C type plate, and Maxi-Soap plate. In addition, the carrier can be of various types and shapes, including plate-like type, such as microtiter plate or disk; granular type, such as beads; tubular type, such as test tubes; fibrous type; membrane-like type; and fine particulate type, such as latex particles. The appropriate carrier will depend upon the assay method.

Beta 2-Glycoprotein I derived from any animal is acceptable, although Beta 2-Glycoprotein I of human origin is preferred. In addition, Beta 2-Glycoprotein I may be prepared in any conventional manner, such as by the method of McNeil, as described in Proc. Natl. Acad. Sci., vol. 87, pages 4120-4128, 1990, which is incorporated herein by reference. Also, the amino acid and nucleotide sequences of Beta 2-Glycoprotein I have been disclosed in Rasmussen et al., “Structure of the human beta2-gycoprotein I (apolipoprotein H) gene”, European Journal of Biochemistry, vol. 259, pp. 435-440 (1999), which is incorporated herein by reference, so preparation by recombinant DNA techniques or peptide synthesis are also acceptable. Furthermore, the sugar chain can be partially or wholly removed and still be used in the present invention. The method of the present invention does not require that Beta 2-Glycoprotein I be highly purified, however, purification is preferred.

As stated earlier, the method of the present invention allows one to “look within” captured immune complexes for specific antigens and antibodies all within a single ELISA test run. Therefore, the four core steps of capturing, dissociating, re-associating, and detecting and quantitating must occur in this specific order. However, other steps of the invention need not occur in a particular order. For example, in an alternative embodiment of the invention, the steps of the invention include: determining the appropriate capture conditions; capturing one or more circulating immune complexes from a sample; adding one or more antigen specific reference materials with enzyme-labeled antigen or antibody to the mixture; dissociating the captured immune complexes by adding dissociating agent; re-associating the captured immune complex with reference material by adding re-associating agent; putting a cover with capture fingers into the wells and incubating; transferring the cover and fingers into substrate wells and incubating; and detecting and quantitating the antigen, antibody, or both antigen and antibody by reading the optical density.

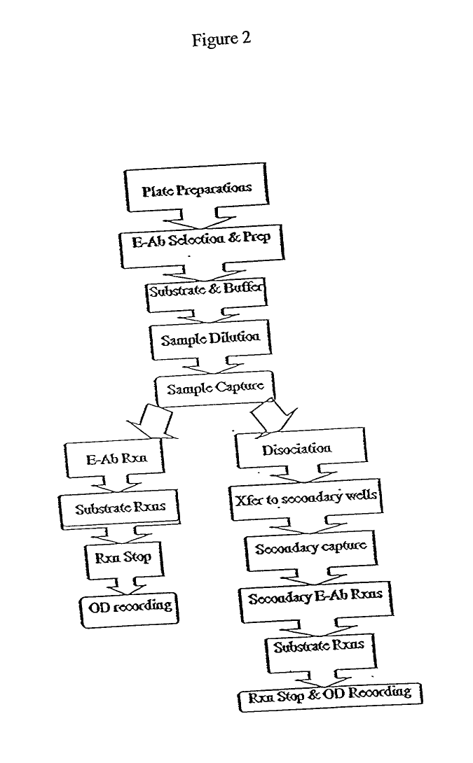

Another embodiment of the method of the present invention is shown schematically in FIG. 2. This figure shows the sequential steps for the testing of intact captured complexes parallel to the testing of dissociated/re-associated complexes. Except in cases where the target to be quantitated is completely masked or saturated, one can detect the target without dissociation, albeit not in a reasonably quantitative manner. In order to determine the relative amount of target, the complexes must be dissociated and re-associated in the presence of the competing labeled marker. It should also be noted that if this method is used as an immuno-proteomic survey tool, then after dissociation the non-immunoglobulin components can be subjected to complete biochemical analysis, or tested against appropriate panels of labeled markers in order to determine what antigen or antigens have solicited a specific humoral immune response.

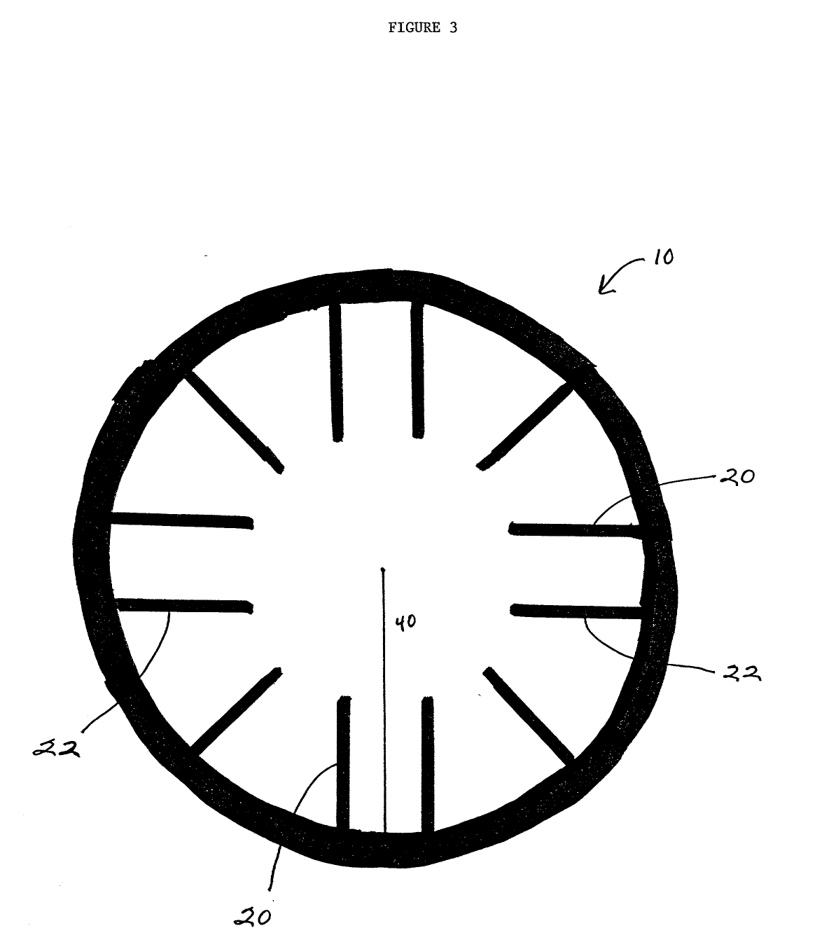

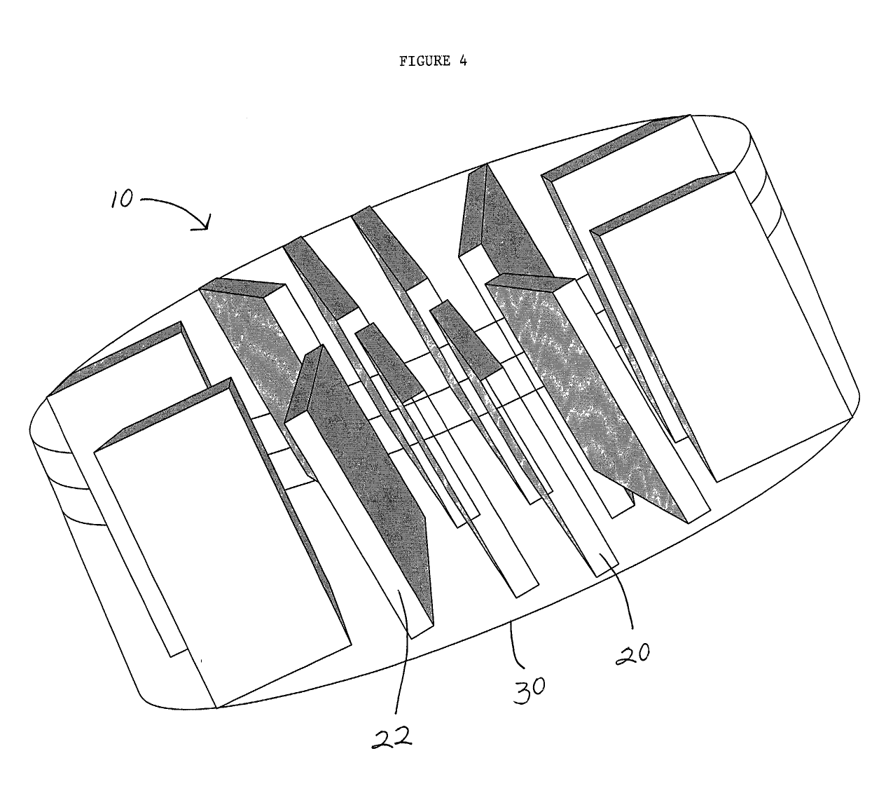

A second aspect of the invention is for a well 10 design that is specifically adapted for use in the method of the invention. One characteristic of the wells 10 are that they have one or more surface area increasing members 20 to increase the well's 10 overall functional surface area. Such members include fins 22, either vertical or horizontal, or any other type of configuration that would increase the overall surface area of the wells 10. Preferably, as shown in FIGS. 3 and 4, the wells 10 would have vertical fins 22 arranged in a radial pattern from the circumference of the well 10 that extend from the well bottom 30 to a height approximately equal to the well radius 40 or about 3% of the height of the well. The fins 22 are in a radial pattern off the circumference of the well 10 in order to increase the liquid-plastic contact area. The number and thickness of the fins 22 are directly related to their ability to increase the effective surface capture area and the ability to facilitate effective washing and rinsing of the well 10. The preferred design would have fins 22 that have a thickness less than the well bottom 30 but greater than 0.25 of the well bottom 30 thickness. In addition, the wells 10 may be made out of any material that will permit the methods of the present invention. However, wells 10 made out of immulon, polystyrene or polyethylene are most preferred.

Another characteristic of the wells 10 is that they are designed to remain substantially optically transparent in the appropriate substrate buffer. Substantially optically transparent means that, upon the addition of buffer, the level of diffraction, interference or absorption in the well will cause a low background in an OD reading that can be normalized or accounted for. The level of background should not be above 0.2, and is preferably below 0.05. Although the surface area increasing members 20 of the wells 10 may have any number of physical configurations, these configurations should not scatter light or cause reflection. Therefore, the surface area increasing members 20 should not be located in the well 10 in a position, or have a configuration that would scatter light when taking an OD reading. By keeping introduced surfaces either at 0 or 90 degrees with respect to the ELISA reader light source, there are no angled surfaces that might lead to the scattering of light or reflection. The purpose of this is so that measurement of an optical density reading will be more accurate.

A third aspect of the invention is for a kit for detecting antigen, antibody, or both antigen and antibody in immune complexes. Each kit will be application dependent, depending upon the particular subset of circulating immune complexes one wishes to capture. Therefore, each kit will contain a potential marker, receptors for the marker, and/or antibodies against any of these. In addition, each kit may contain the appropriate buffers, solutions, wells and other materials necessary to enable a user to perform the method of the present invention. Also, the kits will be designed to take into consideration the states of antigen excess, antibody excess or equivalence based upon the initial pre-screening for which capture method works best. Therefore, the primary purpose of the kit dictates the capture system. For example, if the primary purpose is diagnosis, then Staph proteins could be used to test antigen excess. However, if the primary purpose is therapy, then complement could be used to test antibody excess or equivalence. Furthermore, the kits may contain both positive and negative controls.

The kits are particularly suited for use in the detection and treatment of many types of diseases, including: autoimmune disease, oncology (cancer), and infectious diseases. Specific applications for the kits would involve a host of potential markers, their receptors and/or antibodies against any of these. The markers include coagulation proteins and tumor derived proteins, such as growth factors and their receptors, gene regulation factors, proteins that are important for the development of metastasis, and angiogenic proteins.

Coagulation proteins include, but are not limited to, fibrin degradation proteins (D-dimer, fibrin split products), plasminogen fragments (angiostatin, endostatin), tissue factor, endothelial cell derived proteins and cleaved antithrombin III.

Growth factors and their receptors include, but are not limited to, endothelial growth factor (EGF), Her-2/neu (c-erb-2), c-erb-3 and c-erb-4.

Gene regulation factors include, but are not limited to, p53 protein, c-myc protein and cyclins.

Proteins that are important for the development of metastasis include, but are not limited to, cathepsin D, urokinase plasminogen activator (uPA), tissue plasminogen activator (tPA), matrix metalloproteinasees (MMP), NM23 gene and laminin.

Angiogenic proteins include, but are not limited to, vascular endothelial growth factor (VEGF), basic fibroblastic growth factor (bFGF), platelet derived growth factor (PDGF) and transforming growth factor-β(TGF-β).

Other proteins that are of importance include, but are not limited to, heat shock proteins (hsp) and PS2 protein.

A table of potential antigen targets follows:

| Disease or condition | Receptors | Antibodies |
|---|---|---|
| Anti Phospholipid | Beta 2 Glycoprotein | Antibodies against |
| Syndrome (APS) | | receptors or markers |
| Thrombosis, | Thrombomodulin | Antibodies against |
| Inflammation | | receptors or markers |
| Insulin Dependent | Hsp65 | Antibodies against |
| Diabetes Mellitus | | receptors or markers |
| Sudden Infant Death | MAG | Antibodies against |
| Syndrome | | receptors or markers |
| Tumor Markers | Phospholipase A₂, her- | Antibodies against |
| | 2/neu (c-erb-2), c-erb-3, | receptors or markers |
| | c-erb-4, CEA, PSA, CA | |
| | series, EGF, MAGE-l, | |
| | p53, T/Tn, Muc-1, c-myc | |
| | protein, cyclins, | |
| | cathepsin D, urokinase | |
| | plasminogen activator | |
| | (uPA), tissue plasminogen | |
| | activator (tPA), matrix | |
| | metalloproteinases (MMP), | |
| | NM23 gene, Laminin, | |
| | vascular endothelial growth | |
| | factor (VEGF), basic | |
| | fibroblastic growth factor | |
| | (bFGF), platelet derived | |
| | growth factor (PDGF), | |
| | transforming growth factor | |
| | -β(TGF-β), heat shock | |
| | proteins (hsp), PS2 protein, | |
| | fibrin degradation proteins | |
| | (D-dimer, fibrin split | |
| | products), plasminogen | |
| | fragments (angiostatin, | |
| | endostatin), tissue factor | |
| | endothelial cell derived | |
| | proteins, cleaved | |
| | antithrombin III, d-dimer | |
| | and C-reactive protein. | |
| Crohn's Disease | Phospholipase A₂ | Antibodies against |
| | | receptors or markers |
| Carcinoma | AMF | Antibodies against |
| | | receptors or markers |
| Systemic Lupus | DNA | Antibodies against |
| Erythematosus & | | receptors or markers |
| Renal Insufficiency | | |
| Rheumatoid Arthritis | Rheumatoid Factor | Antibodies against |
| | | receptors or markers |

If, however, a marker for a disease was not known, the marker could be characterized by the method described previously on pages 8-9 of the present application. Therefore, any present or new marker associated with a particular condition may be put into a kit.

A series of tests and procedures were conducted to utilize the method of the present invention.

### Materials

Wash/diluent stock - phosphate buffered saline (PBS) with 0.05% bovine serum albumin (BSA).

Stop solution - a HC1 solution with a concentration greater than or equal to 1N.

Type II sterilized de-ionized water

A spectrophotometer capable of reading the absorbance at 490 nm of samples having a volume of 100-200 μl.

### EXAMPLE 1

### Sample Collection and Preparation.

Sample blood was obtained by venipuncture. After the sample was obtained, it was allowed to clot for 2 hours at room temperature. The sample was then refrigerated for 15 minutes. Following refrigeration, the sample was centrifuged for 5 minutes at 4,000 G and the supernatant (sera) was removed. The sera can be stored at 2-4° C. if used within 48 hours. However, if long term storage of the sera is needed, it should be stored at −70° C. Care should be taken to avoid the introduction of ethylenediamine tetraacetic acid (EDTA) and/or other chelating agents because they may affect the binding of the various components of the immune complexes. The exact volume of sera to be tested can be chosen by the end user, but should be normalized for any one test. However, while the sensitivity of the assay may be increased proportionally to the volume of sample utilized, practical limitations do exist, such as the absolute volume of the well. Therefore, the recommended volume of sera per test sample is 100 microliters (μl). In addition, all samples should be diluted to give a dilution or a range of dilutions that fall within the range of the standard curve. It is recommended that the patient samples be diluted 1:50. A 1:50 dilution is produced by adding 1.45 ml of wash/diluent solution to 50 μl of prepared sera.

### EXAMPLE 2

### Positive Controls.

The positive control has been standardized to allow for development of a standard curve for the evaluation of the test samples. Lyophilized artificially produced immune complexes are stabilized in human serum. Therefore, 250 μl of sterile de-ionized type II water was added to lyophilized human antigen immunoglobulin or Heat Aggregated Human IgG (HAI) stock. After reconstitution, there was approximately 500 μg equivalent/ml in phosphate buffered saline (PBS) with 0.05% bovine serum albumin (BSA) having a pH of approximately 7.2. The positive control, an undiluted solution at 500 μg equivalent/ml concentration, was then diluted to generate the standard curve.

### EXAMPLE 3

### Negative Control.

The negative control should score in the normal patient range when properly employed in the assay. Lyophilized human serum negative control which should score in the normal patient range was reconstituted in 300 μl of sterile de-ionized type II water to produce an undiluted negative control. After reconstitution, the negative control was diluted 1:50 with the wash/diluent solution. A 1:50 dilution is produced by adding 1.45 ml of wash/diluent solution to 50 μl of the reconstituted negative control. The negative control was then run as a test sample.

### EXAMPLE 4

### Plotting the Standard Curve.

The concentrations of immune complexes in the patient samples were read from a standard curve. In order to be as precise and accurate as possible, a standard curve should be produced for each round of testing.

To develop a standard curve and determine the microgram equivalent/ml for each patient sample, the positive control of example 2 was diluted using the wash/diluent solution. The positive control was diluted to the following six concentrations: 62.5, 31.25, 15.625, 7.8125, 1.9531 and 0 μg/ml. The 62.5 μg equivalent/ml solution was produced by adding 100 μl of the reconstituted positive control (HAI stock) to 700 μl of wash/diluent solution. Next, the 31.25 μg equivalent/ml solution was produced by adding 400 μl of the 62.5 μg solution to 400 μl of wash/diluent solution. The remaining concentrations were produced by continuing the 1:2 serial dilution using 400 μl of the previous solution with 400 μl of the wash/diluent solution.

Next, the OD 490 nm readings for the positive control dilutions were plotted against the appropriate μg equivalent/ml on semi-log paper. Then, a straight line was drawn that best fits the plotted points. This is the standard curve. If a single point caused an anomalous deformity in the shape of the curve, the point was omitted. If more than one point lied outside the normal expected curve, then the assay was repeated.

Experiments have indicated that this standard curve is optimized between 2 and 60 μg equivalents/ml. Using this standard curve, find the point corresponding to the 1/25 dilution of each patient sample read at 490 nm and record the corresponding μg equivalent/ml as the value for that sample. If the 1/25 dilution results in a reading over the top of the curve, then a 1/75 dilution reading should be used for determining the μg equivalent/ml result. The corresponding μg equivalent/ml result for the 1/75 dilution should be multiplied by a factor of 3, equaling the quantitative μg equivalent/ml result.

However, if quantitation is not desired, or is unnecessary, and a significant difference above negative control is desired, one can substitute the above procedure with a concentration of 10 μg equivalent/ml of positive control. This should result in a positive control reading in excess of 0.5 OD at 490 nm, which is quite visible, and capable of offering visual comparison of samples and controls.

NOTE - with age or extended multiple uses, a slight flattening of the curve may be observed. This should not create any major difficulty provided that the values obtained for the controls remain within the normal range.

### Expected Results.

The test values showed that:

A. Any sample recording below 2.5 μg equivalent/ml should be considered normal. A mean value of 1.8 μg equivalent/ml should be expected for a normal patient sample with a standard deviation of 1.5.

B. Any sample recording between 2.5 and 4.4 μg equivalent/ml should be considered a borderline result such that further testing should be conducted.

C. Any sample recording between 4.4 and 30 μg equivalent/ml should be considered elevated above normal.

D. Any sample recording in excess of 30 μg equivalent/ml should be diluted further and tested in the assay again.

With this being said, it should be realized that in particular situations, or within particular patient populations, the user may wish to establish a different baseline for normalcy.

### Limitations for Using a Spectrophotometer.

It is important to know the spectrophotometer's upper limit for linearity for absorbance at 490 nm. Many such devices are not linear above readings of 1.5. Whenever a reading is obtained that is beyond the spectrophotometer's upper limit of linearity, a sufficient volume of the solution must be obtained and diluted such that its OD 490 falls within the limits of linearity. Failure to perform this additional dilution and reading in some models could result in an unacceptable standard deviation for quantitating such solutions. Thus, manufacturers directions must be observed. Where additional dilutions are needed, the user need only multiply by the dilution factor employed to normalize the reading with the other readings generated during the assay.

It is recommended that samples and controls are tested in duplicate. If so, the OD values should be averaged.

It is important to note that if the spectrophotometer is zeroed on an empty unused well, then a reading for the negative control in excess of 0.4 OD or a standard curve which reads over 0.8 OD would indicate that a complete set of tests and controls must be repeated.

### EXAMPLE 5

### Method of Pre-Treating Wells with Cross-Linker.

Cyanogen bromide: pre-treatment of wells with cross-linkers followed standard procedures for cyanogen bromide, as disclosed in Johnstone & Thorpe, “Immunochemistry in Practice”, Blackwell Scientific Publications, pp. 205-208 (1982), which is incorporated herein by reference. Essentially, 15.0 gm of cyanogen bromide solution was slowly added to 20.0 ml of 0.5 M carbonate buffer while 4 N NaOH was used dropwise to keep the pH between 10.5 and 11.0. 200 μl of this solution was immediately applied to micro-titer wells and allowed to incubate for 10 minutes at room temperature. The liquid was carefully decanted, and the wells were washed at least 10 times with 0.1 M cold citrate buffer having a pH of approximately 6.5 with a 2-3 minute equalizing phase of citrate buffer in the wells at each wash. 150-200 μl of the material to be bound to the wells, approximately 5- 10 mg/ml for immunoglobulin in citrate buffer, was then added and the wells were sealed with parafilm and refrigerated for 8-12 hours. The wells were then decanted and 250 μl of 2 M ethanolamine was added to each well for 1 hour at room temperature. Next, the wells were decanted and washed at least 3 times with cold PBS. NOTE - all work involving cyanogen bromide and anything that comes into contact with it must be carried out in a fume hood due to its volatility and toxicity!

Gluteraldehyde: approximately 200 μl of 0.1% gluteraldehyde was pipetted into micro-titer ELISA wells and incubated for 1 hour at room temperature. The wells were washed at least 6 times with cold 0.18 M PBS, decanted, and allowed to drain inverted for 5 minutes prior to use.

Poly-L-lysine: approximately 200 μl of 2-5 mg/ml solution of poly-L-lysine was pipetted into micro-titer ELISA wells and incubated for 1 hour at room temperature. The wells were washed at least 6 times with cold 0.18 M PBS, decanted, and allowed to drain inverted for 5 minutes prior to use.

All of the treated wells were further “blocked” with 1% BSA in PBS solution for at least 2 hours in the refrigerator after incubation with the appropriate capture molecule.

### EXAMPLE 6

### Well Preparation.

Plate preparations involve coating the interior of the wells with molecules capable of capturing/binding the desired circulating immune complex set or subset. For example, Staphylococcal proteins can be used for general collection. Also, Clq, anti-Clq, C3d, anti-C3d, conglutinnin, or Raji cell membrane extracts may be used to coat the interior of the wells. In addition, the wells may be blocked with bovine serum albumin prior to lyophilization. After lyophilization, the wells are ready for use, i.e., there is no need to rehydrate the wells prior to use.

Wells were prepared containing lyophilized Clq on the bottom of each well. The wells were prepared by incubating 150 μl of a 10 μg/ml solution of Clq in the wells for 15 minutes at room temperature. The wells were emptied prior to lyophilization. A solution of 1% RIA grade BSA was used to block the wells.

### EXAMPLE 7

### Well Preparation Using Staph Proteins.

Wells containing Staph proteins were also prepared. Staphylococcus Aureus protein preparations, Protein A and Protein G, were purchased from Pharmacia Corp., Piscataway, N.J. 150-200 μl of the appropriate protein preparation, at approximately 20-100 μg/ml, were applied to the wells for 1 hour at room temperature. The wells were then washed 3 separate times with PBS. The wells were then incubated for 1 hour with 1% BSA in PBS to block, followed by one more wash.

Alternatively, whole Staphylococcus Aureus may be employed by following the methods described in Johnstone & Thorpe, Immunochemistry in Practice, pp. 209-210 (1982), or Agnello, Human Pathology, vol. 14, pp. 343-349 (1983), both of which are incorporated herein by reference.

### EXAMPLE 8

### Well Preparation Using Raji Cell Membrane Preps.

Wells with Raji cells were prepared. Raji cell membrane preps were purchased from ATC, Cherry Hill, N.J. 150-200 μl of the Raji cell prep at approximately 20-100 μg/ml was applied to the wells for 1 hour at room temperature. The wells were then washed 3 separate times with PBS. The wells were then incubated for 1 hour with 1% BSA in PBS to block, followed by one more wash.

Raji cell membrane preps can also be used to prepare wells by following the methods described in Coyle et al., “A micro ELISA Raji cell assay to detect immune complexes”, Journal of Immunological Methods, vol. 74, pp. 191-197 (1984).

NOTE --- Each of the following three embodiments of the invention, examples 9, 10 and 11, may be referenced from either: Agnello, V., “Immune Complex Assays in Rheumatic Diseases”, Human Pathology, vol. 14, pp. 343-349 (1983), or Lambert et al., “A WHO collaborative study for the evaluation of eighteen methods for detecting immune complexes in serum”, Journal Clinical Laboratory Immunology, vol. 1. pp.1-15 (1978), which are incorporated herein by reference. The major modification of the present invention deals with stopping the ELISA formats outlined above and holding the bound circulating immune complex in the wells for further use. However, other modifications are described above and also in the examples below. In addition, the methods of the present invention have some minor additional modifications, such as the use of fragmented cell membranes as a capture molecule instead of prepared or purified molecules, that can be found in Coyle et al., “A micro ELISA Raji cell assay to detect immune complexes”, Journal Immunogical Methods, vol. 74, pp. 191-197 (1984), which is incorporated herein by reference.

### EXAMPLE 9

### Method of Capturing CICs Using Complement Component Clq.

100 μl of diluted samples and/or prepared dilutions of positive and negative controls were each added to a previously prepared well and incubated for 1-2 hours at room temperature (23° C.). The previously prepared well was prepared according to example 5. The incubations were followed by three separate washing steps with wash/diluent solution. The wash/diluent solution remained in the wells for 3 minutes for each washing step. The wells should not remain without liquid for prolonged periods of time because this drying may deleteriously affect the performance of various biological materials.

### EXAMPLE 10

### Method of Capturing CICs Using Staph Proteins.

The procedure of example 9 was followed, except that the wells were prepared according to example 6.

### EXAMPLE 11

### Method of Capturing CICs Using Raji Cell Membrane Preps.

The procedure of example 9 was followed, except that the wells were prepared according to example 7.

### EXAMPLE 12

### Method for Detecting CICs with Polyethylene Glycol (PEG).

PEG was added to a solution containing both monomeric and complexed immunoglobulin (Ig) to produce a final concentration of 2% PEG. At 2% PEG, complexed immunoglobulin was selectively precipitated, with free immunoglobulin remaining in the supernatant. Centrifugation was used to pellet the precipitate. The supernatant was then decanted. The precipitate was subsequently washed with 2% PEG in PBS. Next, the precipitate was redissolved in PBS so that the complexed immunoglobulin could be quantitated.

### EXAMPLE 13

### Method of Secondary Capture.

Either of two different methods may be employed. In the first method, the initial capture system was utilized and the same wells used for dissociation and re-association. The enzyme-labeled reactant was then introduced such that the enzyme-labeled reactant and the target molecule formed a new circulating immune complex, which was then captured. The plates were then washed to remove the unbound materials. After adding substrate, OD readings were taken. The OD readings related to the amount of target antigen in the sample.

In the second method, dissociation takes place in the well with the captured circulating immune complex. Re-association with the enzyme-labeled reactant takes place in the same well However, material was either withdrawn into new wells that were set up for capture or an additional capture surface was introduced into the wells, such as ELISA coverplates with “dipsticks”.

### EXAMPLE 14

### Method of Dissociating Captured Immune Complexes Using High Salt Concentration.

This was tested in 2 discrete steps.

In the first step, a synthetic preparation of circulating immune complex containing human IgG and murine IgM-anti-human IgG, also known as heat aggregated immunoglobulin (immune complex without the antigen), purchased from Sigma Chemical, St. Louis, Mo., was dissolved in physiological saline to reach a concentration greater than 125 mg/ml. The synthetic immune complex was prepared by combining each immunoglobulin source (human IgG and murine IgM-anti-human IgG) at molar equivalence for 1 hour at room temperature. The particulates were then centrifuged out at low speeds, and the solution was passed through a Sephadex column. The 3 recovered bands were tested for the presence of each component. The heaviest band was the only one which showed the presence of both human and murine immunoglobulin. Concentrations were determined spectrophotometricly.

The solution was then diluted 1:4 into a dissociating buffer containing 500 mM NaCl, 2 mM EDTA, and 50 mM sodium phosphate at pH 7.3 and 30.5 mmho (milli mho, units of conductance) at approximately 23° C. This solution was then passed through a Sephadex column and 2 bands were identified by absorbance at 280 nm. The bands were collected, dialyzed against PBS overnight, and tested for the presence of human IgG or murine IgM by latex bead agglutination assays with latex beads coated with either anti-human IgG or anti-murine IgM. Each band was found to have only one component, thus suggesting separation, as the original preparation in PBS led to only one band when passed through the Sephadex column.

In a follow-up experiment, immune complexes were bound in wells for each of the previously outlined ELISA systems. The complexes were washed free of unbound materials and the excess liquid shaken free. A high salt solution containing 500 mM NaCl, 2 mM EDTA, and 50 mM sodium phosphate or 50 mM Tris at pH 7.3 at approximately 23° C. was added to the wells in various volumes and for various times. This high salt solution worked best with the Clq and Staphylococcal protein capture systems. It also worked, but not as well, with the Raji cell membrane system as there appeared to be less material recovered from the Raji based capture system.

### EXAMPLE 15

### Method of Dissociating Captured Immune Complexes Using Low pH.

A model circulating immune complex solution was made by mixing molar equivalent solutions of human IgG and murine IgM-anti-human IgG, purchased from Sigma Chemical, St. Louis, Mo., in physiological saline to reach a concentration greater than 2 mg/ml. This solution was prepared by combining each immunoglobulin source at molar equivalence for 1 hour at room temperature. The particulates were centrifuged out by low-speed centrifugation and collected. The solution was then eluted through a Sephadex G-25 column, purchased from Pharmacia Corp., Piscataway, N.J., with physiological saline. The 3 recovered bands were tested for the presence of each component. The first band, containing larger molecules, was the only one that showed the presence of both human and murine immunoglobulin. Concentrations were determined spectrophotometrically. The particulate precipitates, which occurred only when mixtures of the immunoglobulin existed, were also considered to be composed of both immunoglobulins.

Different acid treatments were applied to the precipitated circulating immune complex pellets. 0.5 M HCI, acetic acid, or citric acid, all purchased from Fischer Scientific, Chicago, Ill., were added dropwise into 1 ml of the CIC precipitates in physiological saline until the pH was less than 2.5, but greater than 1.5. This generally required 1 or 2 drops of acid. In all cases, the visible precipitates were solubilized, indicating dissociation of the complexes, at least to some degree.

The bands from the G-25 column were diluted 1:2 in immunoglobulin depleted plasma, i.e., normal human plasma that had been passed through a Protein-A/G Sephadex column, purchased from Pharmacia Corp., Piscataway, N.J., and 100 μl was added to the wells in each of the capture systems. ELISA measurements were recorded for each system and normalized to a value of one hundred percent for that capture system. The wells from each ELISA system were stopped prior to secondary labeled antibody stages, and were treated with 20 μl of acid treated saline with a pH of approximately 2.5 or with 20 μl of normal PBS. After 10-30 seconds of acid/control exposure, the wells were treated with 100 μl of secondary enzyme-labeled anti-Ig, purchased from Sigma Chemical, St. Louis, Mo., in 1% BSA in PBS having a pH of 7.2. The well's entire contents were transferred to identical capture system wells that had not been subjected to any acid treatments.

The sample treatments and ELISA results are as follows:

| ELISA SYSTEMS | C1q CIC | Staph CIC | Raji CIC |
|---|---|---|---|
| SAMPLE TREATMENTS | % | % | % |
| Negative control - plasma | 0 | 0 | 0 |
| Untreated CIC band | 100 | 100 | 100 |
| Untreated human IgG band | 2 | 6 | 3 |
| Untreated murine IgM band | 1 | 2 | 2 |
| Acid CIC band | 3 | 10 | 6 |
| Acid human IgG band | 2 | 1 | 3 |
| Acid murine IgM band | 1 | 2 | 2 |

These results were interpreted to mean that acid treatment of captured circulating immune complexes dissociated the immune complex, and the secondary antibody prep diluted the immune complex sufficiently to effectively prevent reformation and recapture of the immune complex. The low level readings found were thought to be the result of the secondary antibody complexing to some degree with the immune complex or their components, and being themselves captured as labeled antibody, mixed directly with either the human IgG, murine IgM or CIC bands.

### EXAMPLE 16

### Method of Re-Associating Captured Immune Complexes with Reference Materials by Decreasing the Salt Concentration.

Two methods, dilution and dialysis, both worked but are not preferred because of the greater time and equipment needs required.

### EXAMPLE 17

### Method of Re-Associating Captured Immune Complexes with Reference Materials by Increasing the pH.

0.5 ml of the acid dissociated CIC preparations (pellets) from example 14 were treated with either 0.5 ml of PBS at a pH of 7.2 or with 0.5 ml of tris-HCl at a pH of 8.0, and incubated at room temperature for 60 minutes. Turbidity was present but gentle centrifugation for 10 minutes yielded a visible pellet that was similar in size and nature to the initial precipitate pellet. This result indicates that acid dissociation could be reversed by returning the pH to over 6.0 within 30-120 seconds of the initial acid exposure. However, as the time of acid treatment exceeded 30 seconds, the amount of the recoverable pellet decreased, and the nature of the pellet was visibly altered.

As in example 14, bands representing circulating immune complexes, human IgG and murine IgM were independently applied to ELISA systems, except for a few differences. The first difference was that the capturing molecules were cross-linked into the ELISA wells. The second difference was that some wells were not exposed to acid treatment; some wells were exposed to acid treatment alone; and other wells were exposed to acid treatment, followed by treatment with a base, 1 drop of 0.5 M NaOH, to restore the pH to approximately 7.2. In addition to transferring materials after treatment with acid and pH restoration into new wells, other wells saw no removal of materials with the ELISA proceeding as before. While the OD readings varied between the different types of cross-linkers, the relative readings within each system were remarkably consistent. A typical cross-linker response is shown instead of all three. The CIC control response was set to equal 100% for each capture system. The results are as follows:

| | ELISA SYSTEMS | C1q CIC | Staph CIC | Raji CIC |
|---|---|---|---|---|
| | SAMPLE TREATMENTS | % | % | % |
| No | Negative control − plasma | 0 | 0 | 0 |
| Volume | Plasma + acid | 0 | 0 | 0 |
| transfer | Plasma + acid + base | 0 | 0 | 0 |
| | *Untreated CIC band | 100 | 100 | 100 |
| | Untreated human IgG band | 3 | 7 | 3 |
| | Untreated murine IgM band | 2 | 3 | 4 |
| | Acid CIC band | 3 | 9 | 5 |
| | Acid human IgG band | 1 | 2 | 3 |
| | Acid murine IgM band | 3 | 1 | 2 |
| | A + B CIC band | 58 | 95 | 85 |
| | A + B human IgG band | 2 | 4 | 5 |
| | A + B murine IgM band | 4 | 8 | 8 |
| WELL | Negative control − plasma | 0 | 0 | 0 |
| Volume | Plasma +acid | 0 | 0 | 0 |
| trans- | Plasma + acid + base | 0 | 0 | 0 |
| ferred | *Untreated CIC band | 100 | 100 | 100 |
| | Untreated human IgG band | 1 | 4 | 2 |
| | Untreated murine IgM band | 2 | 1 | 2 |
| | Acid CIC band | 1 | 5 | 3 |
| | Acid human IgG band | 1 | 2 | 2 |
| | Acid murine IgM band | 1 | 1 | 1 |
| | A + B CIC band | 48 | 93 | 81 |
| | A + B human IgG band | 2 | 2 | 3 |
| | A + B murine IgM band | 3 | 1 | 2 |

*These groups were normalized per capture system

These results indicated that circulating immune complexes can be dissociated and re-associated within the same ELISA well and retain measurability, at least in a relative sense, providing that the acid treatment is reversed within about 30 seconds and sufficient complement exists in the samples.

### EXAMPLE 18

### Preparation of Enzyme-Labeled Antibody and Substrate Buffer.

Enzyme-labeled antibody preparations were purchased from Sigma Chemical, St. Louis, Mo., with alkaline phosphatase or horseradish peroxidase as the enzyme-substrate system. The antibodies were directed to either human immunoglobulin heavy chains (to reflect the presence of circulating immune complexes), Beta 2 Glycoprotein, various other anti-cardiolipin antibodies, or enzyme-labeled cardiolipins.

Lyophilized anti-human immunoglobulin was complexed to the enzyme horseradish peroxidase. In this particular example, lyophilized horseradish peroxidase labeled goat anti-human immunoglobulin was added to 325 μl of sterile de-ionized type II water. This stock was then diluted by addition of 10 ml of wash/diluent solution. The diluted solution was then ready for use.

The substrate for horseradish peroxidase, O-phenylenediamine (OPD), was dissolved in 12.5 ml of phosphate-citrate buffer. Therefore, no hydrogen peroxide was needed. The phosphate-citrate buffer blocked much of the non-specific background to allow a greater sensitivity and specificity for the enzyme reaction. As a result, OPD which acts as the substrate for the horseradish peroxidase of the conjugate produced a quantifiable orange-brown color. After the stock has been prepared, it should be kept between 2-6° C. and should not be exposed to light. This solution should also not be re-used.

### EXAMPLE 19

### Reaction with Anti-Human Immunoglobulin Enzyme Conjugate.

After the samples were incubated in the prepared wells and the wells were washed, the wash solution was decanted and the wells inverted. The wells were then tapped to remove any remaining wash solution. Next, reference material, 100 μl of prepared horseradish peroxidase-linked anti-human immunoglobulin, was added to the wells and incubated for 1 hour at room temperature. The reference material was in a solution of PBS at a pH between 6.8 and 7.4. The incubations were followed by three separate washing steps with the wash/diluent solution. The wash/diluent solution remained in the wells for 3 minutes for each washing step. The wells should not remain without liquid for prolonged periods of time because this drying may deleteriously affect the performance of various biological materials.

### EXAMPLE 20

### Substrate Reaction.

After completing the step of adding the enzyme-labeled conjugate that is illustrated in the previous example, the fluid was decanted from the well and the well inverted. The plate was then tapped several times to remove any remaining solution. 100 μl of the substrate solution (OPD dissolved in phosphate citrate buffer) was then added to each well. The incubation should proceed for 30 minutes at room temperature. The incubation should also occur in an environment that is protected from exposure to light. The well was wrapped in aluminum foil. Alternatively, the wells could be placed in a box.

The user should limit exposure of this solution to light because the substrate solution is light sensitive. In addition, the solution should be made immediately prior to use and should not be re-used.

The incubation time is also important. Decreasing the incubation time will decrease the overall readings, with the background being decreased to a greater extent than the positive readings. On the other hand, increasing the incubation time may increase the apparent sensitivity of the assay, but is often complicated by a concurrent increase in the background readings.

### EXAMPLE 21

### Stopping the Enzyme-Substrate Reaction.

After the 30 minute incubation reaction, 20 μl of stop solution was added to each well. Our stop solution comprised hydrochloric acid at a concentration of 1N. Alternatively, the substrate reaction could be stopped by adding 25 μl of 2% hydrogen peroxide in PBS.

### EXAMPLE 22

### Recording the Results.

The results should be read on a standard ELISA reader at the appropriate optical density (OD) for the enzyme-substrate system.

A plate reader was used to record the results. The plate reader was zeroed on an empty well. The wells were then read and the optical densities recorded at 490 nm for each patient sample on a patient log. This should occur within one hour of stopping the substrate reaction.

### EXAMPLE 23

### Method for the Competitive ELISA.

The method for the competitive ELISA is described in Voller et al., “The Enzyme Linked Immunosorbent Assay (ELISA)” Dynatech Labs catalogue, no. 011-010-6200.

### EXAMPLE 24

### Detection of Hidden or Masked CIC Components.

The method of the present invention is also able to detect hidden or masked components of circulating immune complexes. First, a series of immune complexes were constructed such that one component of the complex was available in decreasing amounts. Next, polyethylene glycol (PEG) precipitation was used to collect the immune complexes from the free immunoglobulins. This was followed by placing the re-dissolved PEG precipitates (immune complexes) in ELISA wells coated with murine anti-human IgM. After a 2 hour incubation, the wells were washed and an enzyme-labeled secondary antibody against human IgM and human IgG were added to identical samples. OD readings of the samples and controls were taken. The results obtained for the equimolar CIC precipitates were normalized to 100%. A review of the overall procedure, along with some modifications, is described in Agnello V., “Immune Complex Assays in Rheumatic Diseases”, Human Pathology, vol. 14, pp. 343-348 (1983), which is incorporated herein by reference.

Human Rheumatoid Factor (RF-IgM), purchased from Sigma Chemical, St. Louis, Mo., was reacted with pooled human IgG, also purchased from Sigma, starting at equimolar concentrations and then titrating the IgG twofold. The experiment was started with 2 ml of 1 mg/ml RF solution. The RF and IgG solutions were both in PBS at a pH of 7.2, and allowed to incubate at room temperature for 1 hour. PEG was then slowly added to a final concentration of 2%. The resulting solution was centrifuged at approximately 2000 G for 10 minutes to pellet the precipitate. The supernatant was discarded and the pellet was washed 3 times with 2% PEG in PBS. The pellet was then resolubilized in 1 ml of PBS having a pH of 7.2 by vortexing vigorously. Samples from each resolubilized CIC precipitate were then added to ELISA wells that were coated with either murine anti-human IgM or murine anti-human IgG. After incubation, the wells were washed. This was followed by the addition of a secondary enzyme-labeled antibody against either human IgG or human IgM, depending on what the wells were coated with. Next, substrate was added and subsequently stopped. Finally, OD readings were taken.

### Sample & Ratio of RF:IgG

A1=1:1

A2=2:1

A3=4:1

A4=8:1

A5=16:1

A6=32:1

C1=RF alone

C2=IgG alone

C3=PBS

Solutions were centrifuged and the pellets were collected, washed and resuspended in PBS. 100 μl of each pellet preparation was added to wells coated with either anti-human IgM (M capture system) or anti-human IgG (G capture system). Secondary enzyme-labeled antibody was directed to either human IgM (EM) or human IgG (EG). After washing and substrate incubation, OD readings were taken with all results being normalized to 100%.

The ELISA results were as follows:

| | |
|---|---|
| M Capture System | G Capture System |

| Sample | E^{M} | E^{G} | E^{M} | E^{G} |
|---|---|---|---|---|
| A1 | 100% | 100% | 100% | 100% |
| A2 | 112 | 86 | 102 | 90 |
| A3 | 83 | 47 | 91 | 68 |
| A4 | 37 | 12 | 28 | 12 |
| A5 | 20 | 2 | 5 | 2 |
| A6 | 18 | 1 | 2 | 1 |
| C1 | NP | NP | NP | NP |
| C2 | NP | NP | NP | NP |
| C3 | 0 | 0 | 0 | 0 |

NP means no pellets were obtained. Therefore, no ELISA readings were possible. These data would seem to suggest that as the RF:IgG ratio approaches 16:1, that less IgG is in the complex and possibly much of the RF is sterically hindering its recognition by the capture and/or the secondary enzyme-antibody.

To test the hypothesis above, samples A5 and A6 were prepared at double the previous pellet concentration by halving the standard volume of PBS that was used to resolubilize the pellet. Sample A1 was prepared in the same fashion as above. The same capture systems were employed as above except the antibodies were cross-linked into the wells with cyanogen bromide as previously described. The use of gluteraldehyde as the cross-linking agent gave similar results. Acid treatment of non-cross-linked capture molecules seemed to dissociate them from the wells. After the circulating immune complexes were captured in the wells, acid dissociation was initiated using HCl. Then, enzyme-labeled secondary antibodies were introduced into the well along with buffer to restore the pH, as previously described. The enzyme-labeled secondary antibodies were of murine origin and previously had been shown not to react with human RF. These secondary antibodies were added in excess, limited only by their ability to lead to non-specific background. The results were as follows:

| M Capture | | | Captured CIC (dissociated & | |
|---|---|---|---|---|
| System | Captured CIC (untreated) | | re-associated) | |
| Sample | E^{M} | E^{G} | E^{M} | *E^{G} |
| A1 | 100% | 100% | 100% | 100% |
| A5 | 48 | 4 | 53 | 18 |
| A6 | 26 | 3 | 37 | 9 |
| C1 | 1 | 3 | 1 | 0 |
| C2 | 2 | 1 | 1 | 2 |
| C3 | 0 | 0 | 0 | 0 |
| G Capture | | | Captured CIC (dissociated & | |
| System | Captured CIC (untreated) | | re-associated) | |
| Sample | E^{M} | E^{G} | E^{M} | E^{G} |
| A1 | 100% | 100% | 100% | 100% |
| A5 | 10 | 3 | 4 | 4 |
| A6 | 5 | 2 | 3 | 2 |
| C1 | 2 | 2 | 1 | 2 |
| C2 | 1 | 3 | 2 | 1 |
| C3 | 0 | 0 | 0 | 0 |

*E^{G}- These re-associated samples were transferred to fresh G capture wells, as the M capture wells were incapable of holding the IgG or the enzyme-labeled antibody. The G capture system was unable to bind significant amounts of circulating immune complexes. Therefore, dissociation and re-association had little effect.

These data suggest that when the ratio of RF to IgG exceeds 16: 1, simple capture of the circulating immune complex by anti-IgG was inhibited. In addition, detecting the presence of IgG, even in the anti-IgM captured circulating immune complexes, was inhibited. However, when the M captured complexes were dissociated and then re-associated in the presence of excess enzyme-labeled antibody, the IgG component within the circulating immune complex could be detected. Therefore, hidden components of circulating immune complexes can be exposed and detected by a controlled dissociation and re-association process within the active ELISA well.

This experiment was then repeated employing Staphylococcal protein A and protein G in Clq capture systems. Rather than using PBS as the diluent for the circulating immune complex, human plasma that had its immunoglobulin fraction removed by Protein A/G chromatography was employed.

The results were as follows:

| C1q Capture | | | Captured CIC (dissociated & | |
|---|---|---|---|---|
| System | Captured CIC (untreated) | | re-associated) | |
| sample | E^{M} | E^{G} | E^{M} | *E^{G} |
| A1 | 100% | 100% | 100% | 100% |
| A5 | 96 | 12 | 102 | 24 |
| A6 | 71 | 8 | 66 | 17 |
| C1 | 2 | 3 | 1 | 1 |
| C2 | 2 | 1 | 0 | 2 |
| C3 | 0 | 0 | 0 | 0 |
| Sta. Capture | | | Captured CIC (dissociated & | |
| System | Captured CIC (untreated) | | re-associated) | |
| sample | E^{M} | E^{G} | E^{M} | E^{G} |
| A1 | 100% | 100% | 100% | 100% |
| A5 | 93 | 10 | 78 | 23 |
| A6 | 54 | 6 | 43 | 16 |
| C1 | 1 | 2 | 3 | 2 |
| C2 | 2 | 0 | 2 | 2 |
| C3 | 0 | 0 | 0 | 0 |

These results demonstrate that masked components within circulating immune complexes can be detected within a typical ELISA CIC format by controlled dissociation and re-association reactions. This was seen by comparing the detectable IgG from samples A5 and A6 when the CIC pellets were not treated versus being treated in the wells.

In addition to the above, it should be recognized that:

Many proteins associated with various diseases have been recognized and their clinical utility in the diagnosis and treatment of disease is just being realized. Proteomics is the science that deals with gene products, namely proteins, and concerns itself with the entire collection of proteins (the proteome) produced by a particular cell or organism.

The method of the present invention focuses on antigens that the immune system recognizes as important in disease. This is relevant because certain proteins may serve as sensitive markers for the early detection of various diseases or for the early detection of recurrent disease. Current diagnostic tests look for free proteins in the blood to confirm a diagnosis. On the other hand, the method of the present invention looks at and within molecular conglomerates in the blood so it is able to see what the patient's immune system is recognizing and responding to in terms of a disease. Therefore, the method of the present invention detects markers that current tests miss and is able to detect initial and recurring tumors earlier with less false positive and negative results. As a result, the method of the present invention will redefine the way science deals with proteins (selective proteomics) and accelerate the development of such products as disease diagnostics, prognostic markers and therapeutics because the method of the present invention is able to detect various proteins earlier and more precisely than currently available diagnostic techniques due to the fact that the method uses the body's ability to be immune based. In addition, the method of the present invention allows the physician to see how well or poorly the patient's immune system is dealing with the disease.

Consider, for example, breast cancer. The currently available diagnostic modalities, such as mammography, MRI and tumor markers, do not meet the needs of breast cancer testing in terms of early detection, response to treatments and in monitoring tumor recurrence. The method of the present invention will help address these shortcoming. First, the method will aid in diagnosis because there are no FDA (Food and Drug Administration) approved tests available to detect breast carcinoma prior to its detection by mammography. Second, the method will aid as a prognostic marker because there is currently no good FDA approved plasma or serum test for predicting a patient's response to chemotherapy, hormonal therapy or radiation therapy. Also, the method aids in monitoring recurrence because there is no test available that reliably detects recurrent breast cancer until the disease is incurable. The method also finds novel tumor antigens because many of the best selling drugs either act by targeting proteins or are proteins.

The current screening methods for breast cancer have a high incidence of false positive results. Because the present invention is able to detect various proteins earlier and more precisely than currently available tests and because the method of the present invention can find evidence of disease in ways existing kits can not, the present invention will have a profound effect on the way diseases are diagnosed, recurrences are detected and molecular therapeutic targets will be discovered. The key to many diseases is early detection so the method of the present invention allows individuals to be treated with drug therapy sooner and more accurately, thereby enhancing a patient's chance for recovery. The method of the present invention will be beneficial to all patient subtypes, but especially beneficial for those younger patients for whom current diagnostic modalities, such as mammograms, are not very sensitive.

Furthermore, the method of the present invention can support a range of blood tests, not only for breast cancer, but for various cancers, and also for autoimmune disorders and infectious diseases.

### EXAMPLE 25

### Feasibility of Discovering Biomarkers Within Circulating Immune Complexes.

Four groups of pooled patient sera (10 patients per group, 0.1 ml per patient) were split into two equal volumes. PEG was then added to one sample to reach a final concentration of 2%. The sample was then incubated for 8-12 hours at 4 ° C. in order to specifically precipitate out circulating immune complexes, as per example 12. The PEG precipitates were then kept in cold borate buffer until used. From the other sample of the pool, 0.1 ml was diluted 1:20 in PBS and then plated into microtiter wells that were previously coated with Human Clq. The Clq captured circulating immune complexes were recovered from the solid phase by washing the individual wells with citric acid (0.05 ml @ pH 2.8), with the wash being immediately added to a PBS collection vial (1 ml total of citric wash added to 3 ml of PBS).

Each recovered CIC preparation (PEG and Clq) per sample was pooled so that PEG and Clq collected circulating immune complexes were present together. The samples were then split into equal volumes. NOTE: PEG precipitates were added directly to the Clq circulating immune complex PBS solution, which rapidly went into solution with gentle agitation. One of the samples was acid treated to dissociate the circulating immune complexes and then immediately diluted 10 fold in PBS in order to inhibit re-association. The other sample was diluted similarly in PBS to normalize the volumes.

Therefore, each group of pooled samples had circulating immune complexes that were recovered by both PEG and Clq. The samples were then either tested intact or after at least partial dissociation for suspected relevant antigens. The sample groups and findings are shown below:

| GROUP | CIC Display | d-dimer | PSA | CEA | CA-125 |
|---|---|---|---|---|---|
| Breast cancer | Intact | ++++ | 0 | 0 | ND |
| | Dissociated | ++++ | 0 | 0 | ND |
| Prostatic cancer | Intact | ND | 500 | 0 | ND |
| | Dissociated | ND | 500 | 0 | ND |
| Ovarian cancer | Intact | ND | 0 | 0 | 460 |
| | Dissociated | ND | 0 | 0 | 820 |
| Colo-rectal cancer | Intact | ND | 0 | 870 | ND |
| | Dissociated | ND | 0 | 1260 | ND |

All numerical readings are in μg/ml and are derived from RIA standard curves for the indicated antigens. ND indicates that the assay was not performed.

The d-dimer assay is a fluorescent slide assay that was performed only on the breast cancer samples. The readings were brighter/higher than any single patient sample ever seen.

It is apparent that some suspected relevant biomarkers are contained within circulating immune complexes, however, not all CIC bound biomarkers are detectable on intact circulating immune complexes. Therefore, the clinical significance of any particular sequestered biomarker needs to be elucidated. Whether additional biomarkers are represented within circulating immune complexes of groups of patients, and what their clinical significance may be, both need to be more thoroughly determined.

I claim:

1. A method for detecting the presence of antigen, antibody, or both antigen and antibody in immune complexes from a sample, comprising:
capturing a circulating immune complex from said sample, resulting in a captured immune complex;
dissociating said captured immune complex to form a dissociated immune complex;
re-associating said dissociated immune complex with a reference material to form a reformed immune complex; and
detecting and quantitating said reference material in said reformed immune complex.

2. The method of claim 1, wherein said capturing further comprises adding a capture agent.

3. The method of claim 2, wherein said capture agent is selected from the group consisting of Staphylococcal proteins, complement component Clq and anti-complement antibodies.

4. The method of claim 3, wherein said Staphylococcal proteins are selected from the group consisting of Protein A and Protein G.

5. The method of claim 1, wherein said capturing further comprises capturing with a carrier.

6. The method of claim 5, wherein said carrier is a well.

7. The method of claim 6, wherein said capturing further comprises pre-treating said well with a cross-linker.

8. The method of claim 7, wherein said cross-linker is selected from the group consisting of cyanogen bromide, gluteraldehyde and poly-L-lysine.

9. The method of claim 1, wherein said sample is selected from the group consisting of blood, tears, saliva, lymph and urine.

10. The method of claim 1, wherein said capturing further comprises binding antibodies directed to a marker.

11. The method of claim 10, wherein said marker is a protein.

12. The method of claim 11, wherein said protein is selected from the group consisting of tumor-derived proteins, growth factors and their receptors, gene regulation factors, proteins important for the development of metastasis, angiogenic proteins and coagulation proteins.

13. The method of claim 10, wherein said marker is selected from the group consisting of Beta 2 Glycoprotein, thrombomodulin, Hsp65, MAG, Phospholipase A₂, her-2/neu, c-erb-3, c-erb-4, CEA, CA series, EGF, MAGE-1, p53, T/Tn, Muc-1, c-myc protein, cyclins, cathepsin D, urokinase plasminogen activator, tissue plasminogen activator, matrix metalloproteinases, NM23 gene, Laminin, vascular endothelial growth factor, basic fibroblastic growth factor, platelet derived growth factor, transforming growth factor-β, heat shock proteins, PS2 protein, D-dimer, angiostatin, endostatin, tissue factor, endothelial cell derived proteins, cleaved antithrombin III, C-reactive protein, AMF, DNA and Rheumatoid Factor.

14. The method of claim 1, wherein said capturing further comprises binding antibodies directed to a marker wherein said marker is unknown, said method further comprising the steps of:
pre-screening a patient sample for the presence of elevated circulating immune complex levels;
capturing said circulating immune complex;
performing blot analysis;
identifying individual blots;
performing statistical evaluation of said blots to identify any correlation between a material in said blot to a clinical parameter for a disease; and
choosing a capture method.

15. The method of claim 1, wherein said capturing said circulating immune complex is an initial capture, wherein said initial capture takes place in a well having a plurality of capture surfaces, and further comprising performing a secondary capture that involves a secondary capture surface.

16. The method of claim 15, wherein said secondary capture proceeds in the same well as the initial capture, utilizing the same capture surface as the initial capture.

17. The method of claim 15, wherein said secondary capture proceeds in the same well as the initial capture, utilizing a different capture surface than the initial capture.

18. The method of claim 17, wherein said different capture surface is selected from the group consisting of a plate cover dipstick, a bead and a membrane.

19. The method of claim 15, wherein said secondary capture proceeds in a different well than the initial capture.

20. The method of claim 1, wherein said dissociating further comprises adding a dissociating agent.

21. The method of claim 20, wherein adding said dissociating agent brings the salt concentration of said mixture to a range of 300-1500 mM.

22. The method of claim 21, wherein said salt concentration is in the range of 300-500 mM NaCl.

23. The method of claim 21, wherein said dissociating agent comprises 500 mM NaOH, 2 mM EDTA, and 50 mM Tris buffer.

24. The method of claim 21, wherein said dissociating agent comprises 500 mM NaOH, 2 mM EDTA, and 50 mM sodium phosphate.

25. The method of claim 20, wherein adding said dissociating agent brings the pH of said mixture to a pH range of 0.0-2.8.

26. The method of claim 25, wherein said pH is in the range of 1.5 to 2.5.

27. The method of claim 25, wherein said pH is in the range of 2.0 to 2.5.

28. The method of claim 15, wherein adding said dissociating agent changes the osmolarity of said mixture.

29. The method of claim 1, wherein said dissociating further comprises sonicating at a frequency of over 130 Kc/sec for more than 30 minutes.

30. The method of claim 1, wherein said dissociating occurs for a period of time less than 5 minutes.

31. The method of claim 30, wherein said period of time is 1-120 seconds.

32. The method of claim 30, wherein said period of time is 5-30 seconds.

33. The method of claim 1, wherein said dissociating includes adding bovine serum albumin, said bovine serum albumin having a concentration ranging from 0.1 to 3%.

34. The method of claim 33, wherein said bovine serum albumin is 1% RIA-grade bovine serum albumin.

35. The method of claim 1, further comprising adding to said sample a metered amount of labeled antigen or antibody after said dissociating.

36. The method of claim 1, wherein said reference material includes enzyme-labeled markers.

37. The method of claim 36, wherein said enzyme-labeled markers are selected from the group consisting of antigen, antibody and immune complexes.

38. The method of claim 1, wherein said re-associating further comprises adding a re-associating agent.

39. The method of claim 38, wherein adding said re-associating agent brings the salt concentration of said mixture to a range of 0-250 mM.

40. The method of claim 39, wherein said salt concentration is greater than 50 mM.

41. The method of claim 38, wherein adding said re-associating agent brings the pH of said mixture to a range of 2.8-14.

42. The method of claim 41, wherein said high pH is approximately 7.2.

43. The method of claim 1, wherein said re-associating further comprises diluting said sample.

44. The method of claim 1, wherein said re-associating further comprises dialyzing said sample.

45. The method of claim 1, wherein said re-associating occurs for 20 minutes to 12 hours.

46. The method of claim 1, wherein said re-associating occurs for 20 minutes to 2 hours.

47. The method of claim 1, wherein said re-associating occurs for 1- 2 hours.

48. The method of claim 1, wherein said re-associating occurs for 8-12 hours at 4-10° C.

49. The method of claim 1, wherein said detecting and quantitating further comprises measuring the optical density.

50. The method of claim 1, wherein said detecting and quantitating further comprises performing a competitive enzyme-linked immunosorbent assay.

51. A method for detecting antigen, antibody, or both antigen and antibody in immune complexes from a sample, comprising:
determining an appropriate capture condition;
capturing a circulating immune complex from said sample, resulting in a captured immune complex;
adding an antigen specific reference material having enzyme labeled with either antigen or antibody;
dissociating said captured immune complex to form a dissociated immune complex;
re-associating said dissociated immune complex with said reference material to form a reformed immune complex;
incubating said sample in a well having a cover with capture fingers;
transferring said cover into a substrate well and incubating said sample; and
detecting and quantitating said reference material in said reformed immune complex.

52. A well for use in a spectrophotometer having a light source, said well comprising one or more surface area increasing members, said well being made of a material that remains substantially optically transparent in an appropriate substrate buffer.

53. The well of claim 52, wherein said one or more surface area increasing members comprises fins, wherein said fins are perpendicular to said light source when taking an optical density measurement.

54. A kit for detecting antigen, antibody, or both antigen and antibody in immune complexes in a mixture, comprising:
a marker; and
a reference material, wherein said reference material is selected from the group consisting of antibodies and receptors.

55. The kit of claim 54, further comprising positive and negative control samples.

56. The kit of claim 54, wherein said marker is unknown yet capable of characterization by a method comprising the steps of:
pre-screening a patient sample for the presence of circulating immune complexes;
capturing said circulating immune complex;
performing blot analysis;
identifying individual blots;
performing statistical evaluation; and
choosing a capture method.

57. The kit of claim 54, wherein said marker is Beta 2 Glycoprotein.

58. The kit of claim 54, wherein said marker is thrombomodulin.

59. The kit of claim 54, wherein said marker is Hsp65.

60. The kit of claim 54, wherein said marker is MAG.

61. The kit of claim 54, wherein said marker is selected from the group consisting of Phospholipase A₂, her-2/neu, c-erb-3, c-erb-4, CEA, PSA, CA series, EGF, MAGE-1, p53, T/Tn, Muc-1, c-myc protein, cyclins, cathepsin D, urokinase plasminogen activator, tissue plasminogen activator, matrix metalloproteinases, NM23 gene, Laminin, vascular endothelial growth factor, basic fibroblastic growth factor, platelet derived growth factor, transforming growth factor-β, heat shock proteins, PS2 protein, D-dimer, angiostatin, endostatin, tissue factor, endothelial cell derived proteins, cleaved antithrombin III and C-reactive protein.

62. The kit of claim 61, wherein said marker is selected from the group consisting of D-dimer, Her-2/neu and any of its related proteins or specific amino acid sequences for the detection of breast cancer.

63. The kit of claim 61, wherein said marker is Prostatic Specific Antigen (PSA) for the detection of prostatic cancer.

64. The kit of claim 61, wherein said marker is Carcinogenic Embryonic Antigen (CEA) for the detection of colorectal cancer.

65. The kit of claim 61, wherein said marker is Carcinogenic Embryonic Antigen (CEA) for the detection of liver cancer.

66. The kit of claim 61, wherein said marker is Carcinogenic Embryonic Antigen (CEA) for the detection of pancreatic cancer.

67. The kit of claim 61, wherein said marker is CA-125 for the detection of ovarian cancer.

68. The kit of claim 54, wherein said marker is Phospholipase A₂ for the detection of Crohn's disease.

69. The kit of claim 54, wherein said marker is AMF for the detection of carcinoma.

70. The kit of claim 54, wherein said marker is DNA for detection of thrombosis or inflammation.

71. The kit of claim 54, wherein said marker is rheumatoid factor for the detection of rheumatoid arthritis.
